(19) United States
(12) Patent Application Publication
(10) Pub. No.: US 2021/0219856 A1
(43) Pub. Date: Jul. 22, 2021

(54) MEASURING DEVICE FOR MEASURING PHYSIOLOGICAL DATA OF A MAMMAL

(71) Applicant: Nedap N.V., Groenlo (NL)

(72) Inventor: Roxie Sabri Romero MULLER, Enschede (NL)

(73) Assignee: Nedap N.V., Groenlo (NL)

(21) Appl. No.: 17/058,859

(22) PCT Filed: May 28, 2019
(86) PCT No.: PCT/NL2019/050306

§ 371 Date: Nov. 25, 2020

(30) Foreign Application Priority Data
May 28, 2018 (NL) 2020993

(51) Int. Cl. A61B 5/024; A61B 5/00

(52) U.S. Cl. CPC A61B 5/02416; A61B 2503/40; A61B 5/6801

(57) ABSTRACT

Measuring device for measuring physiological data of an animal such as a mammal for determining at least one condition parameter of the animal, comprising a first and second module (12,14) which are configured to be worn, in use, by the animal, wherein the first and second module are furthermore configured for them, from a condition of being mechanically separated from each other, to be mechanically connected with each other whereby in the connected condition, in use, between the first and second module is a skin part of the animal, wherein one of the modules comprises a source (122) for emitting electromagnetic radiation such as light (L) and wherein another one of the modules comprises a sensor system (142) for measuring a parameter such as intensity of a fraction (Lm) of the emitted radiation (L) received via that skin part and for generating a measuring signal (SL) which is indicative of the measured parameter, wherein the measuring device is configured such that, in use, the first and second module can possibly have been connected or be connected stably with each other only when they have at least one predetermined mutual orientation or when they have at least one mutual orientation that falls within a predetermined range of mutual orientations.

The invention relates to a measuring device for measuring physiological data of an animal such as a mammal for determining at least one condition parameter of the animal, comprising a first and second module (12, 14) which are configured to be worn, in use, by the animal, wherein the first and second module are furthermore configured for them, from a condition of being mechanically separated from each other, to be mechanically connected with each other whereby in the connected condition, in use, between the first and second module is a skin part of the animal, wherein one of the modules comprises a source (122) for emitting electromagnetic radiation such as light (L) and wherein another of the modules comprises a sensor system (142) for measuring a parameter such as intensity of a fraction (Lm) of the emitted radiation (L) received via that skin part and for generating a measuring signal (S_{L}) which is indicative of the measured parameter.

A device of this type is known from NL2015582.

A problem with such a device is that, in use, the source of one of the modules is not always optimally positioned with respect to the sensor system of the other module. According to the prior art, the first and second module are attached to each other by means of a pin/hole connection whereby, after the first and second modules have been connected with each other, the first and second module can be rotated relative to each other around an axial axis of the pin. Even if the first and second module are attached to each other such that the source is situated approximately opposite the sensor system, it can still happen that, in use, the first and second module rotate relative to each other, for instance in that someone inadvertently touches either of the parts, or in that the first and second module in the process of being attached to each other are still rotated relative to each other unnoticed.

Object of the invention is to provide a solution to the problem mentioned. To that end, the measuring device according to the invention is characterized in that the measuring device is configured such that, in use, the first and second module can possibly have been connected or be connected (stably) with each other only when they have at least one predetermined mutual orientation or when they have at least one mutual orientation that falls within a predetermined range of mutual orientations.

Due to there being at least one mutual orientation where the first and second module can be connected with each other, it is only necessary, during connecting, to choose the right orientation where the first and second module can be connected with each other whereby in connected condition the sensor system and the source are situated opposite each other. Once the first and second module have been connected with each other in the correct manner, the position of the sensor system with respect to the source is maintained, remaining at least substantially unchanged.

In particular, it holds that the first and second module can possibly have been connected or be connected (stably) with each other only when they have one predetermined mutual orientation or when they have one mutual orientation that falls within a predetermined range of mutual orientations. In this manner, also when mechanically interconnecting the first module with the second module, no mistakes can be made anymore and it will always be ensured that the source is situated opposite the sensor system. The above-mentioned range of mutual orientations may be a consequence of tolerances but may also have been provided intentionally so that connecting the first module with the second module does not require absolute precision. It is only necessary that the first module and second module within a limited range take up the proper orientation with respect to each other for them to be connected with each other.

In particular, it holds here that the range is limited in a possible limited turning around an axis which is at least substantially perpendicular to a plane which, in use, extends parallel to sides of the first and second module that face each other.

More particularly, it holds here that the limited turning involves a maximum angle A, where A is less than 15 degrees, preferably less than 10 degrees and more preferably less than 5 degrees.

In particular, it holds furthermore that the measuring device is configured such that if the first and second module, in use, have been attached to each other, the first and second module can move relative to each other in a possible limited translation along an axis which is at least substantially perpendicular to a plane which, in use, extends parallel to sides of the first and second module that face each other. It is thus ensured that also with varying thicknesses of the skin part, the first and second module can be comfortably attached to the animal.

According to a practical embodiment, it holds that the second module is provided with a lead-through opening and the first module is provided with a projecting pin, while, in use, the pin extends through the opening.

In addition to this, it holds in particular that an inner surface of the opening and an outer surface of the pin have a shape such that when an axial axis of the pin and an axial axis of the opening coincide, the pin can have been or be inserted into the opening only within at least one limited continuous range of rotation angles around its axial axis and with respect to the opening.

According to a practical embodiment thereof, it holds that the outer surface is provided with at least one groove which extends in a length direction of the pin while the inner surface is provided with at least one ridge which, in use, extends in the groove and/or that the inner surface is provided with at least one groove which extends in a length direction of the opening while the outer surface is provided with at least one ridge which extends in the length direction of the pin and which, in use, extends in the groove.

For the foregoing exemplary embodiments, it holds that when the first and second module have been connected with each other according to the one possibility or one of the possibilities, the sensor system and the source are situated at least substantially opposite each other, such that radiation from the source is directed to the sensor system (142). As mentioned, it holds preferably that the first and second module can be connected with each other only according to one possibility, so that, also during the actual connecting, that is, during the fitting to the animal, no mistakes can be made and the sensor system and source are always situated at least substantially opposite each other when the first and second module have been connected with each other. Approximately right positioning with respect to each other can be a consequence of tolerances to which the first and second module can be manufactured, but can also be a deliberate result of the design of the measuring device. In the latter case, it is thereby accomplished that, in use, upon attachment to the animal, it is only necessary that the first and second module, when being fitted to the animal, are positioned with respect to each other approximately in the right manner, which does not require absolute infinite precision.

In particular, the measuring device is intended, with a relatively long useful life, to make measuring physiological data, such as the heartbeat of the mammal, possible.

In realization of this, the measuring device provides in particular the possibility of measuring physiological data of a mammal for determining at least one condition parameter of the mammal. The measuring device accordingly comprises a measuring device to be worn by the mammal, having a first module and second module to be disposed mutually on opposite sides opposite a skin part of the mammal. The first module comprises the source for generating the radiation. The second module comprises the sensor unit for measuring an intensity of a fraction of the radiation received via that skin part and delivering a measuring signal which is indicative of the measured value of the radiation. The measuring device is preferably configured for pulsewise activating the source, while the measuring signal is indicative of the value of the intensity measured during the pulsewise activation. The measuring device may furthermore be provided with synchronization means for synchronously activating the source and the second module, while the synchronization means comprise an energy transmitting unit which is part of one of the first and the second module, and comprise a detector which is part of the other one of the first and the second module, while in an operating condition the energy transmitting unit pulsewise generates an electromagnetic field, and the detector receives this field and generates therefrom a supply voltage for use in that other module.

The at least one condition parameter to be determined is, for example, a heartbeat of the animal or a blood pressure of the animal. Also, the condition parameter to be determined can be a blood value, i.e., an indication of a concentration of one or more constituents in the blood. Also, a combination of condition parameters can be established with the measuring device and/or the measuring method.

As in the measuring device the radiation used for the measurement is preferably generated pulsewise, the intensity of the generated radiation can be raised without thereby also increasing the electrical energy consumption. Consequently, raising the intensity used does not need to be accompanied by a decrease of the useful life.

The invention will now be further explained on the basis of the drawings, in which:

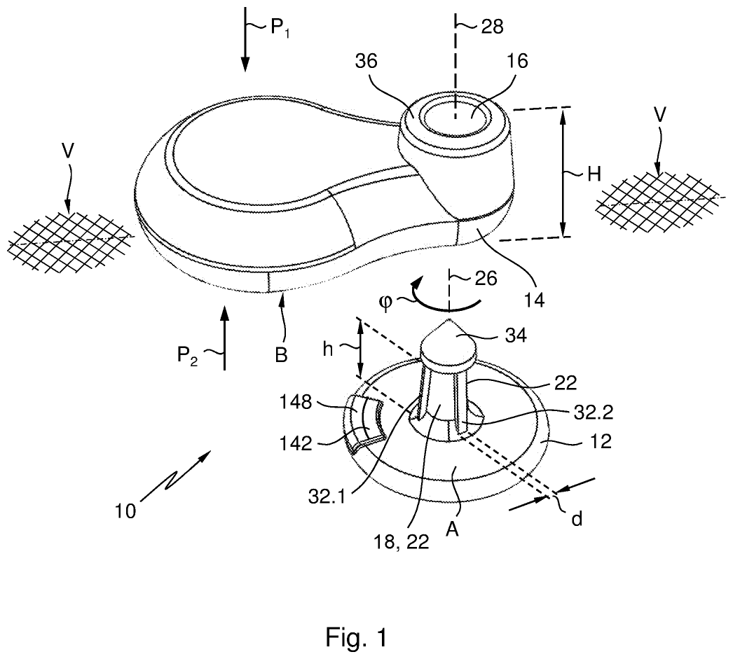

FIG. 1 shows a possible embodiment of a measuring device according to the invention;

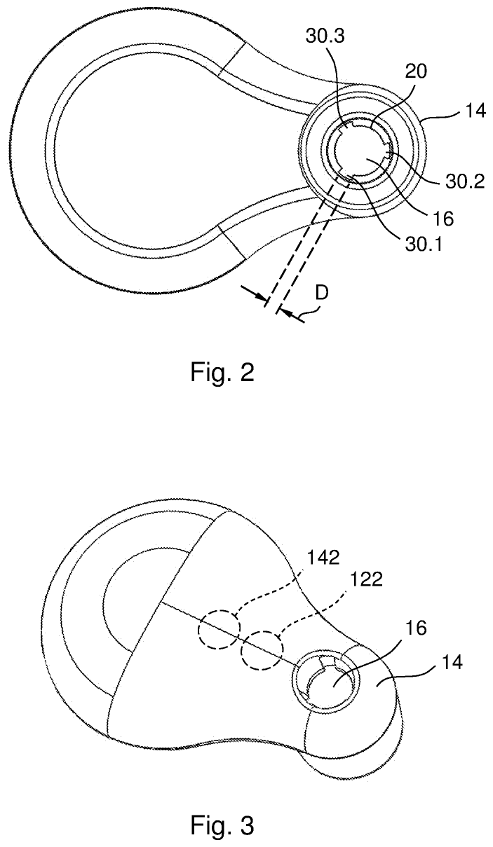

FIG. 2 shows a view of the second module in the direction of the arrow P₁ of FIG. 1;

FIG. 3 shows a view of the second module in the direction of the arrow P₂ of FIG. 1;

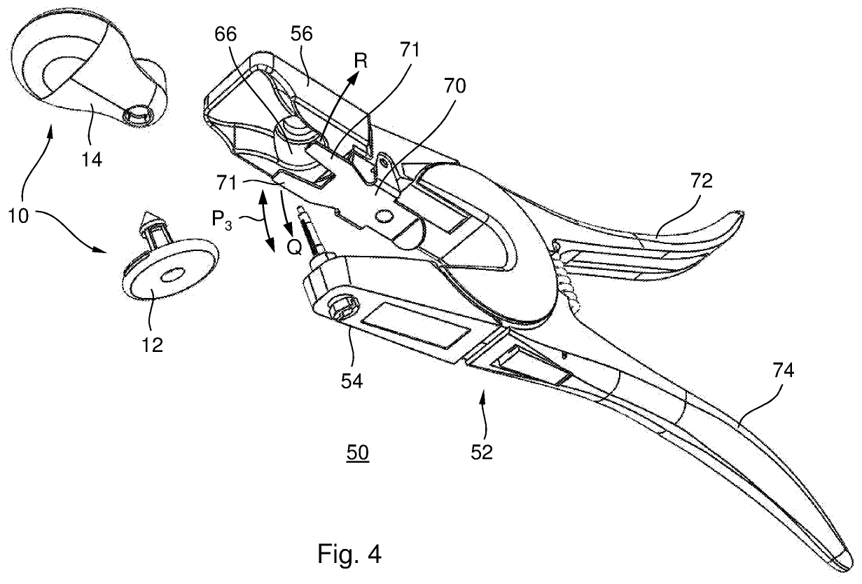

FIG. 4 shows a system according to the invention comprising a measuring device according to the invention and a tool;

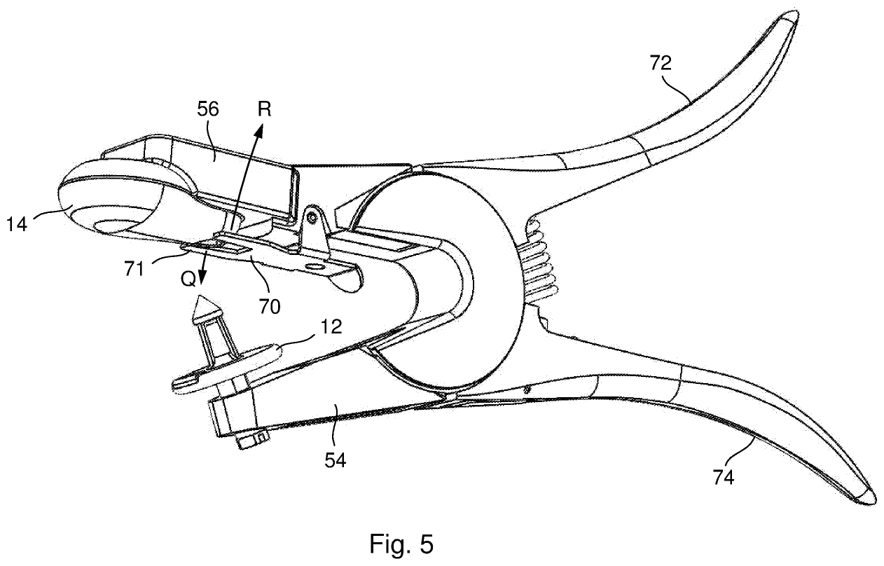

FIG. 5 shows the system of FIG. 4 when the first and second module have been attached to the tool;

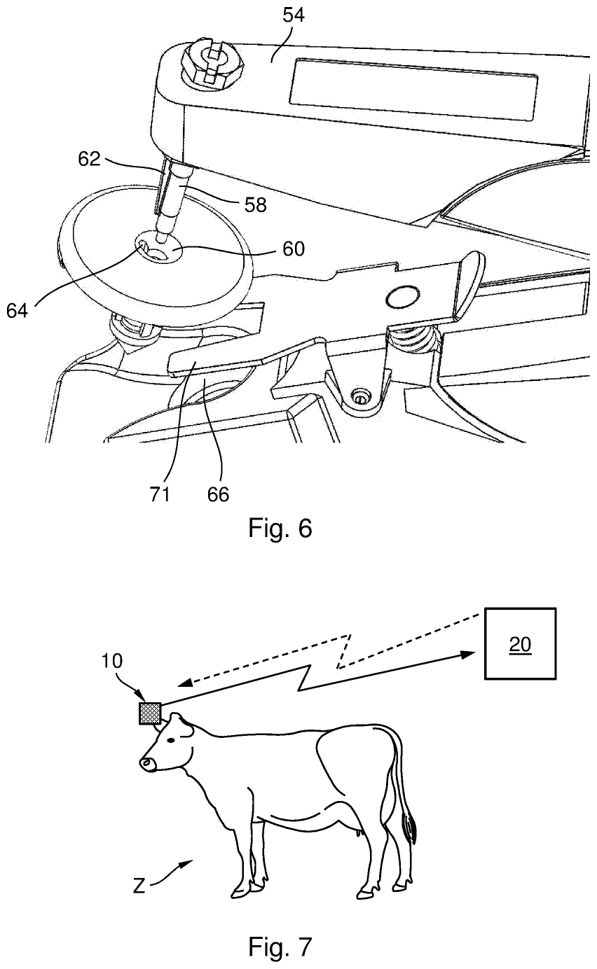

FIG. 6 shows the manner in which the first module can be attached to the tool;

FIG. 7 schematically shows the measuring device of FIG. 1;

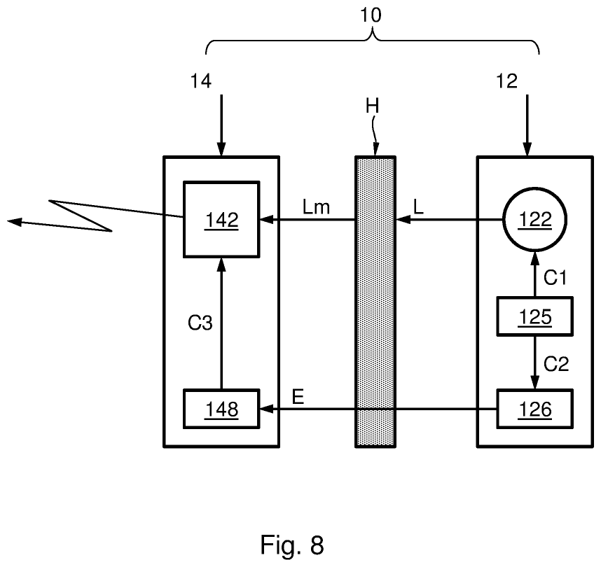

FIG. 8 schematically shows components of the measuring device of FIG. 1;

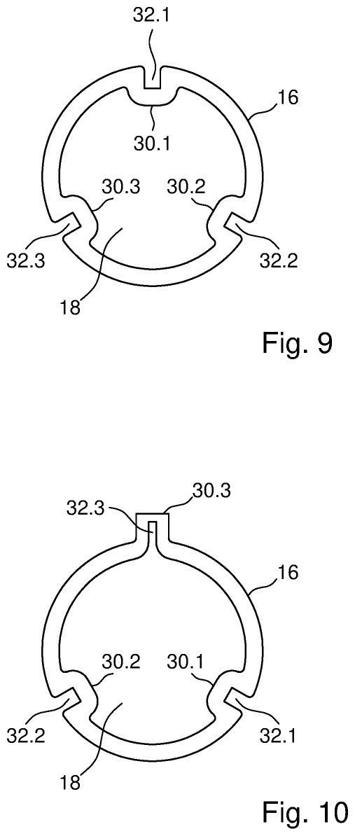

FIG. 9 schematically shows an alternative embodiment of an opening and pin of the device of FIG. 1;

FIG. 10 schematically shows an alternative embodiment of an opening and pin of the device of FIG. 1.

In FIG. 1, with reference numeral 10 an embodiment of a measuring device according to the invention is designated. The measuring device is configured for measuring physiological data of an animal, such as a mammal, for determining at least one condition parameter of the animal. The device comprises a first module 12 and a second module 14 which are configured to be worn, in use, by the animal. The first and second module are configured for them, from a condition of being mechanically separated from each other as shown in FIG. 1, to be connected with each other, whereby in connected condition, in use, between the first and second module is a skin part of the animal, as is also shown in FIG. 8.

One of the two modules comprises a source 122 for emitting electromagnetic rays such as light. In this example, this involves the second module (see FIG. 3). Another one of the modules, in this example the first module 12, is provided with a sensor system 142 for measuring a parameter such as an intensity of a fraction (Lm) of the emitted radiation (L) received via the skin part, and for generating a measuring signal (S_{L}) which is indicative of the measured parameter.

The measuring device 10 is configured such that, in use, the first and second module can possibly have been connected (stably) with each other only when they have at least one predetermined mutual orientation or when they have at least one mutual orientation that falls within a predetermined range of mutual orientations.

In FIG. 1, there is shown the mutual orientation of the first and second module where they can be connected with each other when the first and second module are moved towards each other in the direction of the arrow P₁.

In this example, it holds that the second module is provided with a lead-through opening 16 and the first module 12 is provided with a projecting pin 18, this pin, in use, extending through the opening 16. This situation is shown in FIG. 8.

An inner surface 20 of the opening 16 and an outer surface 22 of the pin 18 have a shape such that when an axial axis 26 of the pin 18 and an axial axis 28 of the opening 16 coincide, the pin can have been or be inserted into the opening only within at least one (in this example three) limited continuous range of rotation angles ϕ around its axial axis and with respect to the opening.

In this example, this has been achieved in that the inner surface 20 is provided with three grooves 30.1-30.3. Further, the outer surface of the pin 18 is provided with three ridges 32.1-32.3 which extend in the length direction of the pin and which, in use, respectively extend in the grooves 30.1-30.3. In this example, this means that the pin can be slid into the opening 16 in three rotational positions, indicated with the angle ϕ. These mutual positions differ by 120 degrees. Preferably, however, it holds that the ridges 32.2 and 32.3 as well as the grooves 30.2 and 30.3 are omitted, so that the pin can only be slid into the opening in one rotational position of the angle ϕ.

If a ridge has a thickness d as shown in FIG. 1 somewhat smaller than a width D of a groove as shown in FIG. 2, this has as a consequence that when the pin has been slid into the opening, it can rotate relative to the opening in a limited continuous range of rotation angles around an axial axis. This has as an advantage that when the pin is being fitted into the opening the ridges and grooves do not need to be perfectly aligned with each other for the pin to be slid into the opening. This applies both to the variant where the opening is provided with three grooves and the pin is provided with three ridges, the ridges being received in the respective grooves, and to the variant where the opening is only provided with a single groove 30.1 and the pin is provided with a corresponding single ridge 32.1. It holds, then, that the first and second module can possibly have been connected with each other only when they have a predetermined mutual orientation. In that case, the width of a groove is approximately equal to the width of a ridge. Or when they have a mutual orientation that falls within a predetermined range of mutual orientations (in that case, the width D of a groove is a bit greater than the thickness d of a ridge). Preferably, it holds in that case that the range is limited in a possible limited turning around the axis which is at least substantially perpendicular to a plane V which, in use, extends parallel to mutually facing sides A and B of the first and second module, respectively. This axis has the same direction as an axial axis 26 of the pin and an axial axis 28 of the opening. The above-mentioned limited turning is due to the ridge having a thickness less than the width of the groove, and involves for example a maximum angle in the direction of the angle ϕ, being less than 15 degrees, preferably less than 10 degrees and more preferably less than 5 degrees.

It holds furthermore that the measuring device is configured such that if the first and second module, in use, have been attached to each other, the first and second module can move relative to each other in a possible limited translation along an axis which is at least substantially perpendicular to a plane V which, in use, extends parallel to sides of the first and second module that face each other.

This limited movement is determined by the difference between a height H of the opening 16 and the height h of the pin up to a knob 34 of the pin. The knob 34 of the pin has a diameter a bit greater than that of the opening 16, such that the knob 34, due to its being made of slightly flexible material, can be forced through the opening 16, whereupon the knob is above an outer surface 36 of the second module. The difference between the height H and the height h determines the distance over which, in use, the first and second module can move relative to each other in the direction of the axial axes 26 and 28. In other words, they can move relative to each other in a possible limited translation along an axis which is, at least substantially, perpendicular to the plane V mentioned.

The possible limited translation is B centimeters at a maximum, where B is less than 6 cm, preferably less than 4 cm, and more preferably less than 2 cm.

In the example shown in FIGS. 1 to 3, then, the first and second module can be connected with each other in three different rotational positions with respect to each other. For one of these positions, then, it holds that the sensor system 142 and the source 122 are situated, at least substantially, opposite each other, such that the radiation from the source is directed to the sensor system. If the grooves 30.2 and 30.3 as well as the ridges 32.2 and 32.3 are omitted, it holds that the first and second module can be connected with each other in one manner only, whereby in that case, in connected condition, the sensor system and the source are situated, at least substantially, opposite each other, such that the radiation of the source is directed at the sensor system.

Preferably, it holds that the measuring device is designed for pulsewise activating the light source, wherein the measuring signal is indicative of the value of the intensity measured during the pulsewise activation, wherein the measuring device is furthermore provided with synchronization means for synchronously activating the light source and the second module (14), wherein the synchronization means comprise an energy transmitting unit (126) which is part of one of the first and the second module and comprise a detector (148) which is part of the other one of the first and the second module, wherein in an operating condition the energy transmitting unit (126) pulsewise generates an electromagnetic field, and the detector receives this field and generates therefrom a supply voltage for use in that other module.

All this will be explained in more detail hereinafter. At this point, however, it is relevant to mention that when the first and second module have been connected with each other according to the one possibility or one of the possibilities, the energy transmitting unit and the detector are situated at least substantially opposite each other, such that the radiation of the energy transmitting unit is directed to the detector.

Again, it holds that when there are three possibilities of attaching the first and second module to each other, it holds only for the possibility where the ridge 32.1 is received in the groove 30.1, the ridge 32.2 is received in the groove 30.2, and the ridge 32.3 is received in the groove 30.3, that the energy transmitting unit and the detector are situated, at least substantially, opposite each other. Again, it holds that if the ridges 32.2 and 32.3 as well as the grooves 30.2 and 30.3 are omitted, there is only one possibility of connecting the first and second module with each other, whereby in this possibility the energy transmitting unit and the detector are situated opposite each other.

Presently, with reference to FIGS. 4-6, a possible embodiment of a system according to the invention will be discussed. The system comprises a measuring device 10 including the first module 12 and the second module 14. The system further comprises a tool 52 including a first part 54 and a second part 56 which are connected movably relative to each other. Viewed from the first part 54, the second part 56 can move along a predetermined path P₃ towards the first part and away from the first part. The system is configured such that the first module 12 can be mechanically connected with the first part in an unequivocal manner. To this end, the tool is provided with a securing pin 58 (see FIG. 6) and the first module 12 is provided with a securing opening 60 in which the securing pin can be inserted for securing the first module to the tool. The securing opening and the securing pin have a complementary shape so that the first module and the tool can be mechanically connected with each other in one manner only.

In this example, this has been realized by providing the securing pin 58 with a ridge 62 and providing the securing opening with a groove 64.

In this example, it holds that the securing opening 60 extends into the pin 18 of a first module to enable the first module to be mechanically connected with the tool in the unequivocal manner. The tool is furthermore provided with a hollow space 66 partly open to the outside, in which a second module can be received for mechanically connecting the second module with the tool in an unequivocal manner. The hollow space 66 and a part of an external side of the second module 14 have a complementary shape so that the second module and the tool can be mechanically connected with each other in one manner only. In this example, the second module 14 can be manipulated in the hollow space 66 and be retained, by means of a spring biased clip 70 and an end 71 of which can be moved by hand in the direction Q so that the second module can be received in the space 66 and of which the free end upon release is moved in the direction R so that the second module is retained in the hollow space 66.

The tool in this example is implemented as a pair of pliers where the first and second part can be moved towards each other by moving two handles 72, 74 respectively towards each other. When the handles 72 and 74 are moved towards each other, the pin 18 will automatically end up in the opening 16, that is, such that the ridge 32.1 is slid into the groove 30.1, the ridge 32.2 is slid into the groove 30.2, and the ridge 32.3 is slid into the groove 30.3. It is noted here that presently it does not matter anymore that in the embodiment in which the first module has three ridges and the second module has three grooves the first and second module can be attached to each other in three different ways (in each case with a difference in rotational position of 120 degrees around the axial axis of the pin), because by the use of the tool only one way of attachment is chosen. As a result of the grooves taking up the ridges, a condition of the first and the second module being stably connected with each other is involved. Accordingly, ‘stably’ is here understood to mean that, after connection, still some limited rotation is possible because of tolerances, or in that this has been determined beforehand and that also in a direction perpendicular to the plane V, that is, in a direction of the axial axis of the pin 18, still a limited translation is possible of the first module relative to the second module to allow the measuring device to be comfortably fitted on the animal.

In FIG. 7 the measuring device is shown in use. Presently, it will be set out how the electronics of the measuring device may be implemented. This is only an exemplary embodiment which stands apart from the idea regarding the manner in which the first and second module can be connected with each other.

As further shown schematically in FIG. 8, the first module 12 includes a light source 122 for generating light L, an energy transmitting unit 126, and a control unit 125 for pulsewise activating the light source 122 and the energy transmitting unit 126.

The second module 14 is provided with a sensor unit 142 and a detector 148. The control unit 125, the energy transmitting unit 126 and the detector 148 jointly form synchronization means for pulsewise activating the sensor unit 142 synchronously with the light source 122. In the first module 12 a battery or other energy source (not shown) is included which supplies the electrical energy for the light source 122, the control unit 125 and the energy transmitting unit 126. The light source 122 is activated with control signal C1. The energy transmitting unit 126, upon activation by the control unit 125 with control signal C2, generates an electromagnetic field E. The detector 148 included in the second module 14 receives this electromagnetic field and generates therefrom a supply voltage, and also control signal C3 with which the sensor unit 142 is driven. In the embodiment shown, the sensor unit 142 and the light source 122 are synchronously activated with the aid of the energy transmitting unit 126 and the detector 148. In other words, the points of time at which the sensor unit 142 and the light source 122 are activated are mutually correlated. As a result, measuring can be done reliably also with a relatively short pulse duration. Moreover, the light source 122 and the sensor unit 142 can be fed from a common energy source. The activation points of time can coincide, but alternatively one of the activation points of time may be shifted by a predetermined time interval relative to the other activation point of time. It may be, for instance, that the sensor unit 142 has a start-up time between the moment of receiving an activation signal C3 from the detector 148 and the moment at which it becomes actually operational. It may also be that the sensor unit 142 after detecting the light pulse Lm needs some time for processing, storage or forwarding information. This can be allowed for in an embodiment in which the control unit 125 activates the control signal C2 in a time interval that begins before the time interval of activating the control signal C1 and that ends following the time interval of activating the control signal C1. Another solution would be for the light source 122 to be activated during a time interval that is long enough for the sensor unit to function properly. This provides the advantage that control is simpler. However, the energy consumption by the light source 122 is higher then.

In the embodiment shown, the light source 122 is an infrared LED or OLED, but if desired an LED may be used for generating light in a different part of the light spectrum, for example, in the visible range, or in the ultraviolet range. Also, conceivably, a different type of light source is used. An LED or OLED, however, is the most suitable for this purpose since this type of light source can be easily driven at relatively low voltages and with a short pulse duration.

The second module 14 has a sensor unit 142 for measuring an intensity of a fraction Lm of the generated light L, received via that skin part, for example the auricle.

The measured intensity depends on inter alia the extent to which light en route from the light source 122 to the sensor unit 142 is absorbed by the blood in the veins and capillaries in that skin part H. This, in turn, depends on inter alia the diameter of the veins and capillaries. The diameter varies periodically with the frequency of the heartbeat. Hence, also the measured intensity varies with that same frequency. In addition, depending on the wavelength of the light, the absorption may depend to a greater or lesser extent on the concentration of constituents in the blood. The sensor unit can thus determine the frequency of the heartbeat as a condition parameter of the mammal from the measured intensity variations.

The invention is not limited to the embodiments described. Thus, the ridges 32.1-32.3 may be provided at an inner side of the opening 16 while the grooves 30.1-30.3 are provided on an outer side of the pin. In use, again, the ridges are received in the grooves as discussed above (FIG. 9). Also, an inner side of the opening may be provided with ridges and grooves while an outer side of the pin is provided with grooves and ridges. In use, the ridges of the pin then fall into the grooves of the opening and the ridges of the opening then fall into the grooves of the pin (FIG. 10). Also, it is possible for the first and second module to be provided with self-locating means which provide that the first and second module when being connected are turned relative to each other in a direction that corresponds with the at least one predetermined mutual orientation or with the predetermined range of mutual orientations. Thus, in a direction in which the pin 18 is inserted into the opening, the grooves may for instance taper to obtain the self-locating effect and hence the self-locating means. As an alternative, it is possible for the ridges to taper in a direction in which the pin 18 is inserted into the opening to obtain the self-locating effect and hence the self-locating means.

1. A measuring device for measuring physiological data of an animal for determining at least one condition parameter of the animal, the measuring device comprising:
a first module and a second module that are configured to be worn, in use, by the animal, wherein the first module and the second module are furthermore arranged to be mechanically connected together, from a condition of being mechanically separated from each other, whereby in the connected condition, in use, between the first and second module is a skin part of the animal, wherein one module of the first and second modules comprises a source for emitting electromagnetic radiation and wherein another of the modules comprises a sensor system for measuring a parameter received via the skin part and for generating a measuring signal which is indicative of the measured parameter, wherein the measuring device is configured such that, in use, the first module and the second module can be connected stably together only when the first module and second module have:
at least one predetermined mutual orientation, or
at least one mutual orientation that falls within a predetermined range of mutual orientations, wherein the predetermined range is limited in a possible limited turning around an axis that is at least substantially perpendicular to a plane that, in use, extends parallel to sides of the first module and the second module that face each other.

2. The measuring device according to claim 1, wherein the first module and the second module can be connected together only at a predetermined mutual orientation or a mutual orientation that falls within a predetermined range of mutual orientation.

3. The measuring device according to claim 1, where A is a magnitude of a maximum angle of the limited turning, wherein A is less than 15 degrees.

4. The measuring device according to claim 1, wherein the measuring device is configured such that after the first module and the second module, in use, have been attached together, the first module and the second module are movable relative to each other in a limited translation along an axis that is at least substantially perpendicular to a plane that, in use, extends parallel to respective facing sides of the first module and the second module.

5. The measuring device according to claim 4, where B is a maximum magnitude of the limited translation, and wherein B is less than 6 cm.

6. The measuring device according to claim 1, wherein the first module and the second module are provided with self-locating structures such that connecting the first module and the second module restricts relative movement of the first module and the second module to turning the first module relative to the second module in a direction that corresponds to the at least one predetermined mutual orientation or to that predetermined range of mutual orientations.

7. The measuring device according to claim 1, wherein the second module is provided with a lead-through opening, and wherein the first module is provided with a projecting pin that, in use, extends through the opening.

8. The measuring device according to claim 7, wherein an inner surface of the opening and an outer surface of the pin have a shape such that when an axial axis of the pin and an axial axis of the opening coincide, the pin is insertable into the opening only within at least one limited continuous range of rotation angle around an axial axis of the pin and with respect to the opening.

9. The measuring device according to claim 8, where A is a magnitude of the continuous range of rotation, and wherein A is less than 15 degrees.

10. The measuring device according to claim 8, wherein:
the outer surface is provided with a groove that extends in a length direction of the pin while the inner surface is provided with a ridge that, in use, extends in the groove, and/or
the inner surface is provided with a groove that extends in a length direction of the opening while the outer surface is provided with a ridge that extends in the length direction of the pin and that, in use, extends in the groove.

11. The measuring device according to claim 1, wherein when the first and second module have been connected together according to one of the possibilities, the sensor system and the source are situated opposite each other such that radiation from the source is received by the sensor system.

12. The measuring device according to claim 1, wherein the measuring device is configured for pulsewise activating the light source, wherein the measuring signal is indicative of the value of the intensity measured during the pulsewise activation, wherein the measuring device is furthermore provided with a synchronizer that facilitates synchronously activating the light source and the second module, wherein the synchronizer includes an energy transmitting unit that is part of one of the first and the second module and comprise a detector that is part of the other one of the first module and the second module, wherein in an operating condition the energy transmitting unit pulsewise generates an electromagnetic field, and wherein the detector receives the electromagnetic field and generates therefrom a supply voltage for use in the other module of the first module and the second module.

13. The measuring device according to claim 12, wherein the measuring device is configured such that, in use, the synchronizer pulsewise activates the light source and the energy transmitting unit, wherein the detector pulsewise activates the sensor system in response to the energy received from the energy transmitting unit or that the synchronizer pulsewise activates the sensor system and the energy transmitting unit, and wherein the detector pulsewise activates the light source in response to the energy received from the energy transmitting unit.

14. The measuring device according to claim 12, wherein when the first module and the second module are connected together according to the one possibility or one of the possibilities, and the energy transmitting unit and the detector are situated at least substantially opposite each other, such that radiation from the energy transmitting unit is directed to the detector.

15. A system comprising:
a measuring device for measuring physiological data of an animal for determining at least one condition parameter of the animal, the measuring device comprising:
a first module and a second module that are configured to be worn, in use, by the animal, wherein the first module and the second module are furthermore arranged to be mechanically connected together, from a condition of being mechanically separated from each other, whereby in the connected condition, in use, between the first and second module is a skin part of the animal, wherein one module of the first and second modules comprises a source for emitting electromagnetic radiation and wherein another of the modules comprises a sensor system for measuring a parameter received via the skin part and for generating a measuring signal which is indicative of the measured parameter, wherein the measuring device is configured such that, in use, the first module and the second module can be connected stably together only when the first module and second module have:
at least one predetermined mutual orientation, or
at least one mutual orientation that falls within a predetermined range of mutual orientations, wherein the predetermined range is limited in a possible limited turning around an axis that is at least substantially perpendicular to a plane that, in use, extends parallel to sides of the first module and the second module that face each other; and
a tool including a first part and a second part that are connected movably relative to each other, wherein, viewed from the first part, the second part is movable along a predetermined path towards the first part and away from the first part,
wherein the system configured such that the first module is mechanically connectable to the first part in one unequivocal manner and the second module is mechanically connectable to the second part in one unequivocal manner,
wherein moving the first part and the second part towards each other causes connecting the first and second module together while the first module and the second:
have the predetermined mutual orientation; or
have the mutual orientation that falls within the predetermined range of mutual orientations.

16. The system according to claim 15, wherein the tool is implemented as a pair of pliers of which the first part and the second part are moveable towards each other by moving two handles of the pliers towards each other.

17. The system according to claim 15, wherein the tool is provided with a securing pin, and wherein the first module is provided with a securing opening in which the securing pin is insertable for securing the first module to the tool.

18. The system according to claim 17, wherein the securing opening extends into the pin of the first module for mechanically connecting the first module with the tool in the unequivocal manner, and
wherein the measuring device is configured such that after the first module and the second module, in use, have been attached together, the first module and the second module are movable relative to each other in a limited translation along an axis that is at least substantially perpendicular to a plane that, in use, extends parallel to respective facing sides of the first module and the second module, and
where B is a maximum magnitude of the limited translation, and wherein B is less than 6 cm.

19. The system according to claim 18, wherein the securing opening and the securing pin have a complementary shape so that the first module and the tool can be mechanically connected together in one manner only.

20. The system according to claim 15, wherein the tool is provided with a hollow space partly open to the outside in which the second module can be received for mechanically connecting the second module with the tool in the unequivocal manner.

21. The system according to claim 20, wherein the hollow space and a part of an outer side of the second module have a complementary shape so that the second module and the tool can be mechanically connected together in one manner only.

22. The tool of the system according to claim 15, with at least a light emitting diode (LED).
